(19) United States
(12) Patent Application Publication
(10) Pub. No.: US 2005/0047957 A1
(43) Pub. Date: Mar. 3, 2005

(54) Autoclave sterilization method and apparatus

(76) Inventor: Donald Nevin, Woodbury, NY (US)

Correspondence Address: EPSTEIN DRANGEL BAZERMAN & JAMES, LLP, 60 EAST 42ND STREET, SUITE 820, NEW YORK, NY, 10165, US

(21) Appl. No.: 10/925,298

(22) Filed: Aug. 23, 2004

Related U.S. Application Data
(60) Provisional application No. 60/499,394, filed on Sep. 3, 2003.

(51) Int. Cl. A61L 2/07; A61L 2/025

(52) U.S. Cl. 422/20; 422/26

(57) ABSTRACT

An instrument sterilization system includes an autoclave with an enclosure into which the instrument to be sterilized is placed. The steam generator of the autoclave is connected to introduce steam into the enclosure. A mechanical energy generator, in the form of ultrasonic generator is connected to a transducer located through the wall of the enclosure. The transducer applies ultrasonic vibrations to the instrument within the enclosure, at the same time as the steam from the steam generator is introduced into the enclosure. The combination of thermal energy and ultrasonic vibrations provides an enhanced ability to eliminate certain harmful prions, viruses and virus precursors on the instruments.

### CROSS-REFERENCE TO RELATED APPLICATIONS

This application relates to the subject matter of Provisional Application No. 60/499,394, filed Sep. 3, 2004, and priority is claimed thereon.

### STATEMENT REGARDING FEDERALLY SPONSORED RESEARCH OR DEVELOPMENT

Not Applicable

### REFERENCE TO A “SEQUENCE LISTING”, A TABLE, OR A COMPUTER PROGRAM LISTING APPENDIX SUBMITTED ON COMPACT DISC

Not Applicable

### BACKGROUND OF THE INVENTION

1. Field of the Invention

The present application relates to techniques for the sterilization of surgical instruments and in particular to an autoclave sterilization method and apparatus which utilizes both thermal energy and mechanical energy in the form of ultrasonic vibrations to improve the ability to eliminate certain harmful prions, slow growing viruses and virus precursors.

2. Description of Prior Art including Information Disclosed Under 37 Cfr 1.97 and 1.98

It is well known that prions, slow growing viruses and virus precursors located on surgical or other medical instruments are not destroyed by conventional steam sterilization autoclave apparatus. Accordingly, certain diseases, and other diseases, may be transmitted using surgical or other medical instruments even after the instruments have been sterilized in a conventional autoclave.

Prion disease is a group of fatal and transmissible neurodegenerative disorders affecting both humans and animals. They include scrapie, chronic wasting disease (CWD) and bovine spongiform encephalopathy (BSE) in animals and Creutzfeldt-Jakob disease (CJD), (commonly known as Mad Cow disease,) Gerstmann-Straussler-Scheinker syndrome and fatal familial insomnia in humans. The unique characteristic of prion disease is its infectivity. The disease can be acquired through experimental inoculation of brain homogenates in humans (hence the occurrence of iatrogenic CJD), and possibly through unintended contamination of animal feces leading to BSE. A great concern is that BSE may have been transmitted to humans and may be responsible for the recent occurrence in many young adults of a new variant form of CJD (vCJD) in the United Kingdom.

The hallmark of prion disease is the presence of an abnormal protease-resistant isoform of prion protein, PrP^{Sc}, in affected brains. A wealth of data supports a central role of PrP^{Sc} in the pathogenesis of prion disease. PrP^{Sc} is thought to derive from the normal cellular prion protein (PrP^{C}) through a conformational change. The two PrP isoforms differ from each other in that PrP^{C} can be easily degraded by treatment with a protease such as proteinase K (PK) while PrP^{Sc} is resistant to PK digestion.

The detection of the protease-resistant PrP has been used as the surrogate marker for the presence of PrP^{Sc} in biological samples. That method is currently the only biochemical technique for definitive diagnosis of prion disease. The most convenient method of this purpose is Western blot analysis using an antibody against PrP after digestion of brain hemogenate with PK. The PK-resistant PrP detected on Western blots provides definitive evidence for the presence of PrP^{Sc}. Several antibodies against PrP, including the widely used 3F4 antibody, are now commercially available. The Western blot immunoassay has shown excellent sensitivity, ease in interpretation and accuracy in definitive diagnosis of CJD.

It is a prime object of this invention to provide a method and an apparatus for eliminating protease-resistant prion proteins, slow growing viruses and virus precursors on surgical instruments through sterilization.

It is another object of the present invention to provide a method and apparatus for eliminating protease-resistant prions proteins, slow growing viruses and virus precursors on surgical instruments by utilizing a combination of thermal energy and mechanical energy in the form of ultrasonic vibrations.

### BRIEF SUMMARY OF THE INVENTION

The present invention relates to an improved sterilization method and apparatus that utilizes both thermal energy and mechanical energy, preferably in the form of ultrasonic vibrations, to break the molecular bonds of the prions, slow growing viruses and virus precursors on surgical instruments to prevent the transmission of disease.

In accordance with one aspect of the present invention, an instrument sterilization device is provided. The device includes enclosure into which the instrument to be sterilized is situated. Means are provided for generating steam. Means are provided for connecting the steam generating means to the enclosure such that steam from the steam generating means is introduced into the enclosure. Means are also provided for generating mechanical energy in the form of ultrasonic vibrations. The ultrasonic vibrations are applied to the instrument within the enclosure at the same time as the steam from the steam generating means is being introduced into the enclosure.

In accordance with another aspect of the present invention, a method for is provided for sterilizing an instrument in an enclosure into which the instrument to be sterilized is situated. The method includes the steps of generating steam and introducing the steam into the enclosure. Mechanical energy in the form of ultrasonic vibrations is generated. The ultrasonic vibrations are applied to the instrument within the enclosure at the same time as the steam is introduced into the enclosure.

### BRIEF DESCRIPTION OF THE SEVERAL VIEWS OF DRAWINGS

To these and to such other objects that may hereinafter appear, the present invention relates to an improved autoclave sterilization method and apparatus, as recited in detail in the following specification and recited in the annexed claims, taken together with the accompanying drawings, wherein like numerals refer to like parts, and in which:

FIG. 1 is a front plan view of the components of the sterilization apparatus of the present invention;

FIG. 2 is a view of the interior of a portion of the interior of the autoclave portion of the apparatus of the present invention; and

FIG. 3 is a side cross-sectional view of a portion of the interior of the autoclave portion of the apparatus of the present invention.

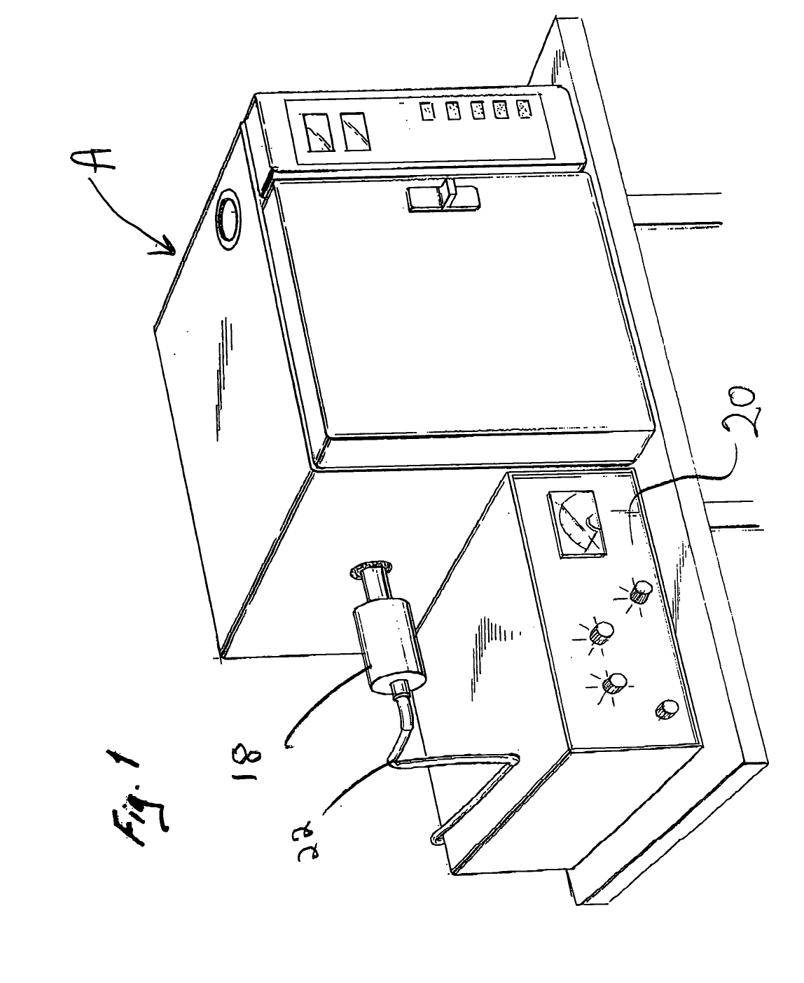

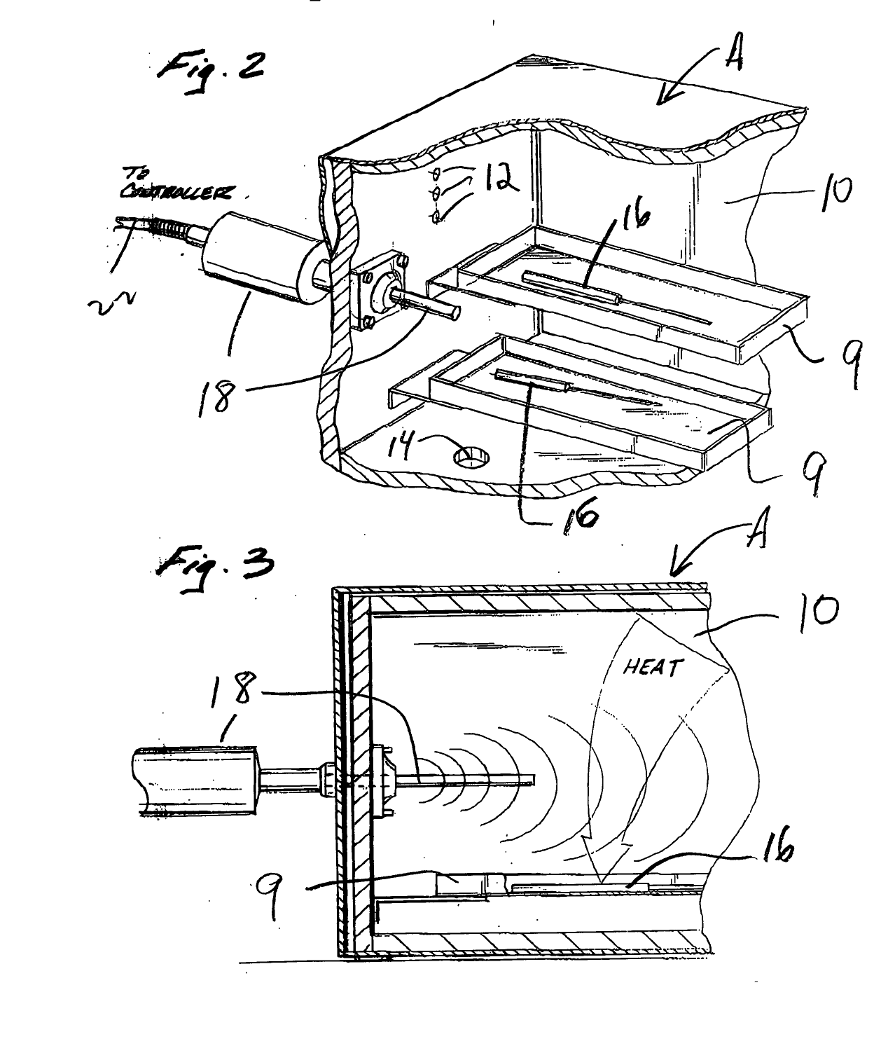

### DETAILED DESCRIPTION OF THE INVENTION

As seen in the accompanying drawings, the method of the present invention is performed in novel apparatus that includes an autoclave, generally designated A, with an internal steam generator (new not shown) a closed steam chamber 10 having one or more steam inlets 12 in the wall of the enclosure connected to the steam generator and one or more outlets or drains 14.

One or more surgical instruments 16 to be sterilized are placed on trays 9 located within in chamber 10. Although a variety of different commercially available autoclaves could be employed to perform the method of the present invention, a Model Validator 8 autoclave from Pelton & Crane of 11727 Fruehauf Drive, Charlotte, N.C. has proven satisfactory for this purpose.

Also situated in operative communication with the interior of chamber 10 of the autoclave is a source of mechanical energy, generally designated B, preferably in the form of an ultrasonic transducer 18. Transducer 18 may extend through the wall or door of the autoclave and may be partially or completely situated within the autoclave chamber.

The energization and control apparatus 20 for the transducer, located outside the autoclave, is connected to transducer 18 by wires 22. Apparatus 20 serves to energize transducer 18 and to control the ultrasonic emissions generated by the transducer. Apparatus 20 may take the form of a Branson “Sonifier” Model No. 250, commonly known as a “cell disruptor”. However, other suitable commercially available ultrasonic energizers may be utilized in this application, as well.

Steam form the internal steam generator of the autoclave is introduced into chamber 10 at the same time as ultrasonic vibrations from transducer 18 are applied to the instruments. By combining the action of the thermal energy from the steam with the effect of the mechanical energy generated by the ultrasonic source, prions, slow growing viruses and virus precursors can be effectively eliminated from surgical instruments, thereby substantially reducing the possibility of disease transmission.

While only a single embodiment of the present invention has been disclosed for purposes of illustration, it is obvious that many variations and modifications could be made thereto. It is intended to cover all of these variations and modifications that fall within the scope of the present invention, as defined by the following claims:

1. An instrument sterilization device comprising an enclosure into which the instrument to be sterilized is situated, means for generating steam, means for connecting said steam generating means to said enclosure such that steam from said steam generating means is introduced into said enclosure, means for generating mechanical energy in the form of ultrasonic vibrations and for applying said ultrasonic vibrations to the instrument within the enclosure, at the same time as the steam from the steam generating means is introduced into said enclosure.

2. An method for sterilizing an instrument in an enclosure into which the instrument to be sterilized is situated, the method comprising the steps of: generating steam, introducing the steam into the enclosure, generating mechanical energy in the form of ultrasonic vibrations, and applying the ultrasonic vibrations to the instrument within the enclosure, at the same time as the steam is being introduced into the enclosure.
